SEQUENCE LISTING (41 sequences)

SEQ ID NO: 1 - protein, 15 aa, Artificial Sequence
  Description of Artificial Sequence Synthetic peptide
FTNPNVKDECGCGES

SEQ ID NO: 2 - protein, 15 aa, Artificial Sequence
  Description of Artificial Sequence Synthetic peptide
HREEEERHKEEKRKR

SEQ ID NO: 3 - protein, 15 aa, Artificial Sequence
  Description of Artificial Sequence Synthetic peptide
HRDDDDRHKDDKRKR

SEQ ID NO: 4 - protein, 11 aa, Artificial Sequence
  Description of Artificial Sequence Synthetic peptide
HREERHKEEKR

SEQ ID NO: 5 - protein, 11 aa, Artificial Sequence
  Description of Artificial Sequence Synthetic peptide
RREEREKEEKR

SEQ ID NO: 6 - protein, 15 aa, Artificial Sequence
  Description of Artificial Sequence Synthetic peptide
KREEEEREKEEKRKR

SEQ ID NO: 7 - protein, 15 aa, Artificial Sequence
  Description of Artificial Sequence Synthetic peptide
HEREREHEKEKREKR

SEQ ID NO: 8 - protein, 15 aa, Artificial Sequence
  Description of Artificial Sequence Synthetic peptide
HREEEERHKEEKRHR

SEQ ID NO: 9 - protein, 15 aa, Artificial Sequence
  Description of Artificial Sequence Synthetic peptide
KEKEKEKEKEKEKEK

SEQ ID NO: 10 - protein, 14 aa, Artificial Sequence
  Description of Artificial Sequence Synthetic peptide
KEKEKEKEKEKEKE

SEQ ID NO: 11 - protein, 13 aa, Artificial Sequence
  Description of Artificial Sequence Synthetic peptide
KEKEKEKEKEKEK

SEQ ID NO: 12 - protein, 12 aa, Artificial Sequence
  Description of Artificial Sequence Synthetic peptide
KEKEKEKEKEKE

SEQ ID NO: 13 - protein, 11 aa, Artificial Sequence
  Description of Artificial Sequence Synthetic peptide
KEKEKEKEKEK

SEQ ID NO: 14 - protein, 10 aa, Artificial Sequence
  Description of Artificial Sequence Synthetic peptide
KEKEKEKEKE

SEQ ID NO: 15 - protein, 9 aa, Artificial Sequence
  Description of Artificial Sequence Synthetic peptide
KEKEKEKEK

SEQ ID NO: 16 - protein, 8 aa, Artificial Sequence
  Description of Artificial Sequence Synthetic peptide
KEKEKEKE

SEQ ID NO: 17 - protein, 7 aa, Artificial Sequence
  Description of Artificial Sequence Synthetic peptide
KEKEKEK

SEQ ID NO: 18 - protein, 6 aa, Artificial Sequence
  Description of Artificial Sequence Synthetic peptide
KEKEKE

SEQ ID NO: 19 - protein, 11 aa, Artificial Sequence
  Description of Artificial Sequence Synthetic peptide
KKEEKKEEKKE

SEQ ID NO: 20 - protein, 11 aa, Artificial Sequence
  Description of Artificial Sequence Synthetic peptide
KKKEEEKKKEE

SEQ ID NO: 21 - protein, 11 aa, Artificial Sequence
  Description of Artificial Sequence Synthetic peptide
KKKKEEEEKKE

SEQ ID NO: 22 - protein, 15 aa, Artificial Sequence
  Description of Artificial Sequence Synthetic peptide
KRKKRRKKKRRRKRK

SEQ ID NO: 23 - protein, 12 aa, Artificial Sequence
  Description of Artificial Sequence Synthetic peptide
HRWCCPGCCKTF

SEQ ID NO: 24 - protein, 12 aa, Artificial Sequence
  Description of Artificial Sequence Synthetic peptide
HRWAAPGAEKTF

SEQ ID NO: 25 - protein, 12 aa, Artificial Sequence
  Description of Artificial Sequence Synthetic peptide
HRWAEPGAEKTF

SEQ ID NO: 26 - protein, 10 aa, Artificial Sequence
  Description of Artificial Sequence Synthetic peptide
QSHEEREHAE

SEQ ID NO: 27 - protein, 13 aa, Artificial Sequence
  Description of Artificial Sequence Synthetic peptide
RERSEEEKEHAEK

SEQ ID NO: 28 - protein, 11 aa, Artificial Sequence
  Description of Artificial Sequence Synthetic peptide
RERSEEEKEHA

SEQ ID NO: 29 - protein, 11 aa, Artificial Sequence
  Description of Artificial Sequence Synthetic peptide
RSEEEKEHAEK

SEQ ID NO: 30 - protein, 9 aa, Artificial Sequence
  Description of Artificial Sequence Synthetic peptide
RSEEEKEHA

SEQ ID NO: 31 - protein, 15 aa, Artificial Sequence
  Description of Artificial Sequence Synthetic peptide
LRLGVRTSGCSGMAY

SEQ ID NO: 32 - protein, 13 aa, Artificial Sequence
  Description of Artificial Sequence Synthetic peptide
LGVRTSGCSGMAY

SEQ ID NO: 33 - protein, 13 aa, Artificial Sequence
  Description of Artificial Sequence Synthetic peptide
LGVRTSGSSGMAY

SEQ ID NO: 34 - protein, 15 aa, Artificial Sequence
  Description of Artificial Sequence Synthetic peptide
FKFTNPNVKDECGCG

SEQ ID NO: 35 - protein, 15 aa, Artificial Sequence
  Description of Artificial Sequence Synthetic peptide
FKFTNPNVKDESGSG

SEQ ID NO: 36 - protein, 15 aa, Artificial Sequence
  Description of Artificial Sequence Synthetic peptide
LHQSREETEHAEKLM

SEQ ID NO: 37 - protein, 10 aa, Artificial Sequence
  Description of Artificial Sequence Synthetic peptide
QSREETEHAE

SEQ ID NO: 38 - protein, 15 aa, Artificial Sequence
  Description of Artificial Sequence Synthetic peptide
SRPYTFHSHGITYYK

SEQ ID NO: 39 - protein, 14 aa, Artificial Sequence
  Description of Artificial Sequence Synthetic peptide
SRPYTFHSHGITYY

SEQ ID NO: 40 - protein, 13 aa, Artificial Sequence
  Description of Artificial Sequence Synthetic peptide
PYTFHSHGITYYK

SEQ ID NO: 41 - protein, 12 aa, Artificial Sequence
  Description of Artificial Sequence Synthetic peptide
PYTFHSHGITYY

(19) United States
(12) Patent Application Publication
(10) Pub. No.: US 2009/0208420 A1
(43) Pub. Date: Aug. 20, 2009

(54) IRON OXIDE-BINDING PEPTIDES

(76) Inventors: Andreas BRIEL, Berlin (DE); Erik Blazek, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/018,429

(22) Filed: Jan. 23, 2008

Related U.S. Application Data
(60) Provisional application No. 60/886,541, filed on Jan. 25, 2007.

(30) Foreign Application Priority Data
Jan. 23, 2007 (DE) 10 2007 004 424.2

(51) Int. Cl. A61K 49/14

(52) U.S. Cl. 424/9.323

(57) ABSTRACT

The invention relates to diagnostic compositions comprising metal oxide particles, especially iron oxide particles, which are coated with binding peptides. The binding peptides bind with high affinity to the metal oxide particles so that the peptide-coated metal oxide particles are suitable for medical applications, for example as contrast media for magnetic resonance imaging.

This application claims the benefit of the filing date of U.S. Provisional Application Ser. No. 60/886,541 filed Jan. 25, 2007.

The invention relates to diagnostic compositions comprising metal oxide particles, especially iron oxide particles, which are coated with binding peptides. The binding peptides bind with high affinity to the metal oxide particles so that the peptide-coated metal oxide particles are suitable for medical applications, for example as contrast media for magnetic resonance imaging.

Superparamagnetic iron oxide nanoparticles (SPIO) composed of magnetite or maghaemite are outstandingly suitable because of their superparamagnetic properties as contrast media for magnetic resonance (MR) imaging.

Beyond the magnetic properties, the suitability of these nanoparticles in practice is substantially determined by physicochemical parameters such as the particle size, the zeta potential and the hydro-phobicity of the surface. The latter are decisive both for the stability in vitro and for the degradation behaviour and distribution in vivo. For these reasons, colloidal preparations of iron oxides have in many cases been provided even during synthesis with a chemical coating which confers the desired properties. Thus, all iron oxide nanoparticles (for parenteral use) which are already in clinical use are provided with a shell of dextran or dextran derivatives, e.g. WO 94/03501. In addition, a large number of organic and inorganic coatings have been investigated (review in [1], e.g. dextran, PEG, chitosan, polyacrylic acid, fatty acids, silanes, silica etc. Silane compounds such as aminopropyltriethoxysilane (APTES) are capable of covalent bonding to iron oxide, but all other coatings are associated with the particles by so-called physisorption (or chemisorption). This non-covalent interaction leads in this case eventually to the formation of an equilibrium between bound and unbound material, depending on the binding strength, i.e. part of the coating is lost after dilution.

SPIO particles are generally broken down by the reticuloendothelial system (RES), the Kupffer cells, in the liver and can therefore be employed primarily for imaging this organ. The half-life in the blood and the uptake, dependent thereon, into the liver can moreover be regulated through the choice of the coating.

In addition, a so-called molecular imaging of clinically relevant target structures in the body, such as, for example, tumours and foci of inflammation, is aimed at by coupling SPIO particles to targeting molecules. Targeting molecules suitable for this task are, besides antibodies and fragments thereof, which can be raised against any desired surface markers, also peptides (review articles [2]) and small molecules such as folic acid [3]. Such contrast media already exist in PET diagnosis, for example the peptide octreotide which is labelled with a radio isotope and binds to a tumour-associated somatostatin receptor. As yet there is no comparable product in clinical use in MR diagnosis. The reason for this is substantially the complicated coupling of the targeting molecules.

A weakly binding coating material such as dextran/carboxydextran is scarcely suitable as carrier of the targeting molecules. Detachment during the course of formation of the binding equilibrium would not only lead to the loss of these molecules, which are in some cases very costly; on the contrary, an attenuation of the signal by blockade of the target structures is also to be expected. Some strategies for more efficient coupling have been worked out in years gone by, for example crosslinking of dextrans, covalent bonding via silanes or coating materials binding with higher affinity.

The object on which the present invention was based was thus to find coating materials for metal oxide particles which avoid at least in part the prior art disadvantages described above. It was intended in particular that the coating materials bind with high affinity and thus with negligible loss of metal oxide particles and, on the other hand, exhibit high biocompatibility.

This object is achieved according to the invention through the provision of a diagnostic composition which comprises a metal oxide particle coated with a binding peptide. The present invention further relates to metal oxide-binding peptides as coating materials and, where appropriate, as molecular anchors for targeting molecules. It has been found according to the invention that peptides can bind to metal oxides such as, for example, iron oxide with higher affinity than is the case with carbohydrates such as, for example, dextran.

The metal oxide particle is preferably a magnetic particle, particularly preferably a superparamagnetic particle. The metal oxide is for example an oxide of a transition metal such as cobalt, nickel, manganese, copper or/and iron. The metal oxide is preferably an iron oxide, e.g. magnetite or maghaemite. The metal oxide particle is preferably a colloidal particle. The average diameter may be in the range 1 nm-1 μm, preferably 1-100 nm and particularly preferably 2-50 nm.

The surface of the metal oxide particle before coating with the binding peptide may where appropriate be modified, e.g. by silane compounds.

The binding peptide used for coating the metal oxide particle normally has a length of up to 100 units, preferably a length of 5-50 units and particularly preferably a length of 5-25 units.

The binding peptide is preferably composed of a linear sequence of amino acids. However, the binding peptide may also comprise one or more modifications of the classical peptide structure, such as, for example, the use of modified amino acid units, alterations in the main chain of the peptide, the cyclization etc. Thus, the units of the peptide may be selected from naturally occurring amino acids, i.e. in particular the genetically encoded amino acids, from non-natural, i.e. artificial non-genetically encoded amino acids, and combinations thereof. The units are preferably L-α-amino carboxylic acids, but it is also possible to use other units such as, for example, D-α-amino carboxylic acids, β-amino carboxylic acids, amino acid analogues etc. Modifications of the main chain include for example replacement of the amide linkage used for connecting the units by other linkages, e.g. N-alkylamide, such as, for example, N-methylamide, ketomethylene, hydroxyethylene, (E)-ethylene, carba, ether, reduced amide, retro-inverso amide, phosphonamide, phosphonate or phosphinate. Further examples of possible modifications of the peptide structure are to be found in Böhm et al., “Wirkstoffdesign”, 1996, Spektrum akademischer Verlag Heidelberg, especially chapter 10.

The binding peptide is preferably prepared by non-enzymatic synthesis, e.g. chemical solid-phase synthesis on a suitable synthetic resin, by known methods. However, in principle an enzymatic synthesis, e.g. in an in vitro translation system or in a cell, is possible.

The binding peptide is selected so that it has an adequate affinity for the metal oxide particle in order to make diagnostic use possible. It was shown by the experiments described in the present application that the binding of peptides to metal oxide particles does not depend on the presence of a specific amino acid sequence.

In a preferred embodiment, the binding peptide comprises a partial sequence of an iron-binding protein such as, for example, ferritin, IscA, ceruloplasmin etc., or a modification of such a sequence, it being possible for example for one, two, three, four or more amino acids to be deleted, added or/and replaced by other units. On the other hand, however, the binding peptide may also have an artificial sequence. Combinations of naturally occurring and artificial sequences are also possible.

Binding peptides comprising at least one basic unit, i.e. a unit with a side chain comprising a basic group, e.g. an amino or guanidino group, such as, for example, arginine (R) or/and lysine (K), may exhibit a particularly high affinity for metal oxide particles. Preferred binding peptides are therefore those comprising one, two, three, four, five or more basic amino acid units. It is further preferred for the binding peptide additionally to comprise at least one acidic unit, i.e. a unit with a side chain which comprises an acidic group, e.g. a carboxylic acid group, such as, for example, glutamic acid (E) or/and aspartic acid (D). The binding peptide preferably comprises one, two, three, four, five or more acidic (amino acid) units. The ratio of basic (amino acid) units to acidic (amino acid) units in the binding peptide is preferably less than 1:2, i.e. less than two acidic units are present for each basic unit. The ratio is particularly preferably less than 1:1.5. It is further preferred for the peptide to comprise one or more hydrophilic (amino acid) units, i.e. units with a side chain which comprises hydrophilic groups, e.g. OH, such as, for example, serine (S).

The isoelectric point of the binding peptide (pI) is typically >5.0, preferably ≧5.5, particularly preferably ≧6.0, even more preferably ≧6.5, even more preferably ≧7.0 and most preferably ≧8.0. Extremely basic peptides with an isoelectric point of up to 13 or more can also bind with high efficiency to metal oxide particles.

The binding peptide favourably comprises both acidic and basic units, e.g. an alternating sequence of positively and negatively charged (amino acid) units which has a particularly high affinity for the surface of the metal oxide particle, on which there are likewise alternating positive and negative charges. A binding peptide which is particularly preferably used is therefore one comprising a sequence:
(BₙAₘ)ᵣ or (AₘBₙ)ᵣ
   where B is in each case independently a basic (amino acid) unit such as, for example, K or R, A is in each case independently an acidic (amino acid) unit such as, for example, E or D,
n and m are each independently 1, 2 or 3, and r is a natural number ≧1, preferably ≧2 and particularly preferably ≧3.

Peptides which are most preferred comprise an alternating sequence of in each case one basic (amino acid) unit and one acidic (amino acid) unit.

The binding peptide may, besides the binding sequence described above which is necessary for binding to the metal oxide particle, also comprise further components such as, for example, spacer molecules, labelling groups such as, for example, dyes, or/and targeting molecules.

### BRIEF DESCRIPTION OF THE DRAWINGS

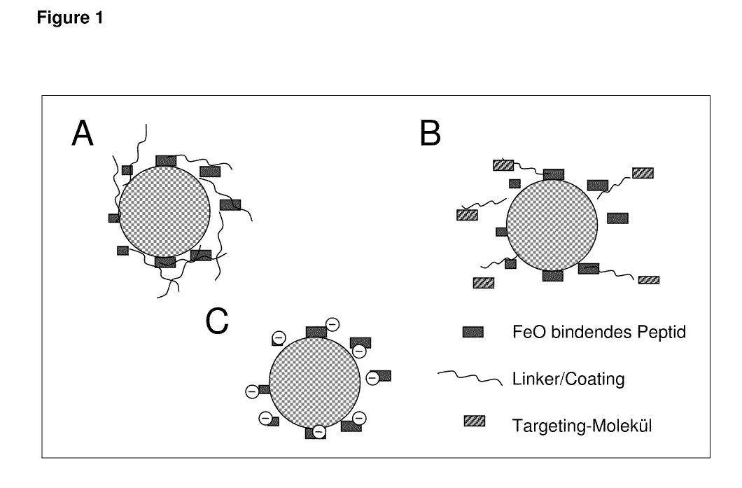

In a first embodiment (see FIG. 1A), spacer molecules can be coupled to the peptide chain. This coupling can take place during or after the peptide synthesis by known methods. Preferred examples of spacer molecules are macromolecules, for example hydrophilic polymers such as, for example, polyalkylene glycols or/and polyalkylene glycol esters, especially polyethylene glycol or/and polyethylene glycol esters, oligo- or polysaccharides, polyalcohols such as polyvinyl alcohol etc. The molecular weight of the spacer molecules is preferably in the range from 500 to 30 000 Da, particularly preferably from 3000 to 5000 Da. The metal oxide particles can be stabilized sterically against aggregation by coupling of spacer molecules, i.e. the spacer molecules act to maintain a distance between the individual particles. It is additionally possible by coating with suitable molecules such as, for example, polyethylene glycol to increase the biocompatibility or/and the circulation time of the particles in the bloodstream.

In a further preferred embodiment, a target-recognition molecule is coupled to the binding molecule (see FIG. 1B). Target-recognition molecules which specifically recognize a target structure in the body, e.g. in particular tissue or cell types, e.g. tumour cells or cells of blood vessels which supply a tumour. The target-recognition molecules may on the one hand themselves be peptide sequences whose sequences can be added even during the synthesis of the binding peptide. Other examples of target-recognition molecules are polypeptides such as, for example, antibodies including antibody fragments, aptamers or low molecular weight compounds. Such molecules can be linked subsequently by known reactions with reactive groups of the binding peptide. Couplings to SH groups may take place for example by using maleimide reagents and to amine groups by using active esters such as, for example, N-hydroxysuccinimide esters. The target-recognition molecules are preferably coupled to the binding peptide via a spacer molecule such as, for example, polyethylene glycol (see FIG. 1B). Suitable coupling methods are described for example in the review article [4].

It is additionally possible by selecting suitable binding peptide sequences to influence the biocompatibility and pharmacokinetics of the metal oxide particle (see FIG. 1C). Thus—in order to increase the bio-compatibility and the circulation time—it is possible to use binding peptides which comprise hydrophilic amino acids such as serine, or/and negatively charged amino acids such as glutamic or aspartic acid.

The dissociation constant of the binding peptide from the metal oxide particle is preferably 10⁻⁶ M or less, particularly preferably 10⁻⁹ M or less. When binding peptides are combined with hydrophilic polymers, it is possible for interactions, e.g. hydrogen bonds, to develop between polymer chains, which bring about an additional anchoring which may additionally enhance the affinity of the coating.

The metal oxide particle may, in addition to the binding peptide, be coated with further materials, e.g. with conventional coating materials such as carbohydrates, e.g. carboxyldextran, polyalkylene glycols such as PEG, polyacrylic or polymethacrylic acids, fatty acids, silica or/and silanes etc. Thus, the metal particle surface can initially be partly coated with peptides which are coupled where appropriate to further components as indicated above, e.g. targeting molecules, and subsequently other coating materials can be applied to the remaining metal oxide surface.

The metal oxide particles according to the invention are preferably employed as diagnostic composition. The use as contrast medium, especially for magnetic resonance imaging, is particularly preferred. The particles can be employed both in veterinary medicine and in human medicine. For a diagnostic use in magnetic resonance imaging, for example 0.001-0.1 mmol of metal, e.g. iron, are administered to a patient per kg of body weight. It is particularly preferred for about 0.01 mmol of iron to be administered per kg of body weight. The administration preferably takes place intravenously. The distribution of the particles in the body is then determined after predetermined times by magnetic resonance or, where appropriate, by other suitable methods. Appropriate methods are known to the skilled person.

Finally, the invention relates to a method for producing a metal oxide particle coated with a binding peptide, comprising:
(a) synthesis of a binding peptide,
(b) where appropriate attachment of a spacer molecule or/and of a target-recognition molecule and
(c) contacting the binding peptide with a metal oxide particle under conditions with which a coating can take place.

The invention is further explained by the following example:

### EXAMPLE

In order to identify iron oxide-binding peptides, 40 different peptides were synthesized in an experiment by chemical synthesis as array on a membrane. Artificial sequences of amino acids, or sequences from proteins of iron metabolism were used in this case. The peptide binding was detected via the absorption of iron oxide particles (more exact information with the preparations) to the membrane.

Commercially available particles (Resovist, Schering AG, Germany—Fluidmag-CT, Chemicell AG, Germany) were used, or particles produced by alkaline shock (standard methods) and destabilized or stabilized with trimethyl-ammonium hydroxide were used. The particles were diluted to an iron concentration of 5 mM in water and incubated with the peptide array at room temperature for 10-20 min. The binding of iron oxide was determined visually, firstly directly and then by forming the colour Prussian Blue with the iron.

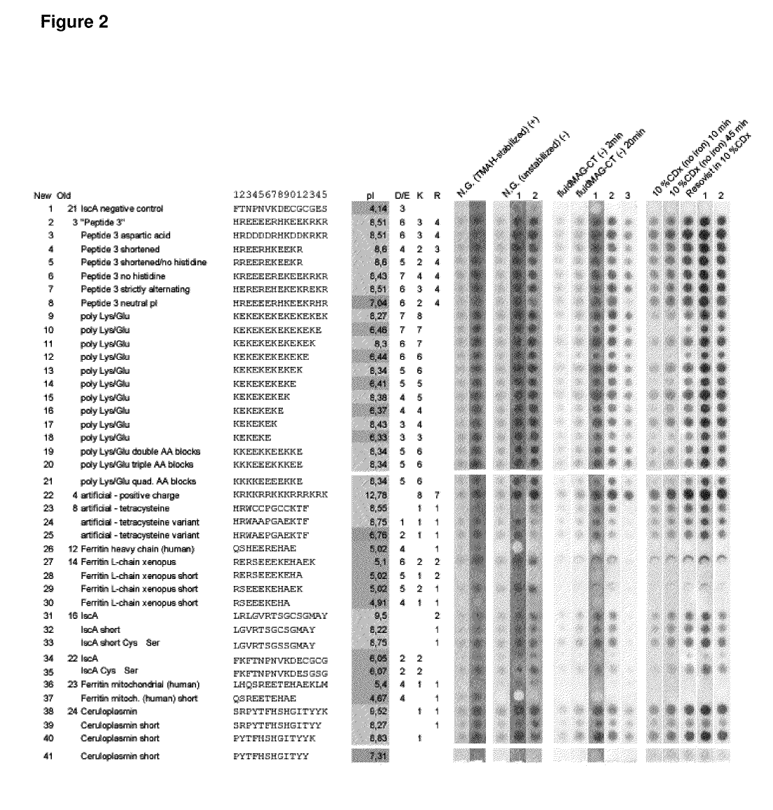

FIG. 2 shows the result of such an experiment. It is evident that peptides 2-25, 27, 29 and 31-40 bring about a binding of iron oxide particles to the membrane, as is evident from the dark coloration.

FIG. 2 discloses SEQ ID NOS: 1-41, respectively in order of appearance.

Peptides 2-21 comprise artificial sequences of negatively charged or acidic and positively charged or basic amino acids and are therefore zwitterionic. Starting from peptide 2, the sequence was modified and simplified until peptides consisting of an alternating sequence of Lys (K) and Glu (E), which likewise bind iron oxide, resulted.

Peptides 26-41 comprise natural sequences from the proteins ferritin, IscA and ceruloplasmin or modifications of such sequences in which, for example, cysteine is replaced by serine. These sequences also show—especially when they have an isoelectric point of ≧5 and in particular of ≧5.5—an efficient binding to iron oxide.

### References:

[1] AK Gupta, M Gupta: Synthesis and surface engineering of iron oxide nanoparticles for biomedical applications. Biomaterials 2005, 26:3995-4021.
[2] J C Reubi: Peptide receptors as molecular targets for cancer diagnosis and therapy. Endocr Rev 2003, 24:389-427.
[3] Y Zhang, N Kohier, M Zhang: Surface modification of superparamagnetic magnetite nanoparticles and their intracellular uptake. Biomaterials 2002, 23:1553-61.
[4] Y Zhang, C Sun, N Kohler, M Zhang: Self-assembled coatings on individual monodisperse magnetite nanoparticles for efficient intracellular uptake. Biomed Microdevices 2004, 6:33-40.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2007 004 424.2, filed Jan. 23, 2007, and U.S. Provisional Application Ser. No. 60/886,541, filed Jan. 25, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Diagnostic composition comprising a metal oxide particle coated with a binding peptide.

2. Composition according to claim 1, characterized in that the metal oxide particle is a magnetic particle.

3. Composition according to claim 1, characterized in that the particle is a superparamagnetic particle.

4. Composition according to any of claim 1, characterized in that the metal oxide particle is an iron oxide particle.

5. Composition according to claim 1, characterized in that the metal oxide particle has an average diameter of from 1 nm to 1 μm, preferably of 1-100 nm.

6. Composition according to claim 1, characterized in that the binding peptide has a length of 5-100 (amino acid) units.

7. Composition according to claim 1, characterized in that the binding peptide comprises natural (amino acid) units, non-natural (amino acid) units and combinations thereof.

8. Composition according to claim 1, characterized in that the binding peptide comprises at least one basic (amino acid) unit, in particular Arg (R) or/and Lys (K).

9. Composition according to claim 8, characterized in that the binding peptide additionally comprises at least one acidic (amino acid) unit, in particular Glu (E) or/and Asp (D).

10. Composition according to claim 8, characterized in that the ratio of basic (amino acid) units to acidic (amino acid) units in the binding peptide is less than 1:2 and preferably less than 1:1.5.

11. Composition according to claim 8, characterized in that the binding peptide additionally comprises at least one hydrophilic (amino acid) unit, especially Ser (S).

12. Composition according to claim 1, characterized in that the binding peptide has an isoelectric point of ≧5.0, preferably of ≧6.0, particularly preferably of ≧7.0 and most preferably of ≧8.0.

13. Diagnostic composition according to any of claim 1, characterized in that a spacer molecule is coupled to the binding peptide.

14. Diagnostic composition according to claim 13, characterized in that the spacer molecule is a hydrophilic polymer, e.g. a polyalkylene glycol or a polyalkylene glycol ester, in particular a polyethylene glycol or a polyethylene glycol ester, an oligo- or polysaccharide or a polyalcohol.

15. Diagnostic composition according to claim 14, characterized in that the spacer molecule has a molecular weight of from 500 to 30 000 Da.

16. Diagnostic composition according to claim 1, characterized in that a target-recognition molecule is coupled to the binding peptide.

17. Diagnostic composition according to claim 16, characterized in that the target-recognition molecule is selected from peptides, polypeptides such as, for example, antibodies, aptamers or low molecular weight compounds.

18. Composition according to claim 1, characterized in that the binding peptide comprises a partial sequence from an iron-binding protein such as, for example, ferritin, Isc A or ceruloplasmin or a modification of such a sequence.

19. Composition according to claim 1, characterized in that the binding peptide comprises an artificial sequence.

20. Composition according to claim 1, characterized in that the binding peptide comprises at least one partial sequence:
(BₙAₘ)ᵣ or (AₘBₙ)ᵣ
where B is in each case independently a basic (amino acid) unit,
A is in each case independently an acidic (amino acid) unit,
n and m are each independently 1, 2 or 3, and
r is a natural number ≧1, preferably ≧2 and particularly preferably ≧3.

21. Composition according to claim 1, characterized in that the dissociation constant of the binding peptide from the metal oxide particle is 10⁻⁶ M or less.

22. Composition according to claim 1, characterized in that the metal oxide particle is, in addition to the binding peptide, coated with further materials.

23. Composition according to claim 1 for use as contrast medium, in particular for magnetic resonance imaging.

24. Metal oxide particle coated with a binding peptide.

25. Method for producing a metal oxide particle coated with a binding peptide, comprising:
(a) synthesis of a binding peptide,
(b) where appropriate attachment of a spacer molecule or/and of a target-recognition molecule and
(c) contacting the binding peptide with a metal oxide particle under conditions with which a coating can take place.
